(19) United States
(12) Patent Application Publication
(10) Pub. No.: US 2024/0394524 A1
(43) Pub. Date: Nov. 28, 2024

(54) WEIGHT ATTENTION FOR TRANSFORMERS IN MEDICAL DECISION MAKING MODELS

(71) Applicant: NEC Laboratories America, Inc., Princeton, NJ (US)

(72) Inventor: Iain Melvin, Princeton, NJ (US)

(21) Appl. No.: 18/670,275

(22) Filed: May 21, 2024

Related U.S. Application Data
(60) Provisional application No. 63/468,053, filed on May 22, 2023.
(60) Provisional application No. 63/525,757, filed on Jul. 10, 2023.

(51) Int. Cl. G06N 3/063; G16H 20/00; G16H 50/20

(52) U.S. Cl. CPC G06N 3/063; G16H 20/00; G16H 50/20

(57) ABSTRACT

Methods and systems for configuring a machine learning model include selecting a head from a set of stored heads, responsive to an input, to implement a layer in a transformer machine learning model. The selected head is copied from persistent storage to active memory. The layer in the transformer machine learning model is executed on the input using the selected head to generate an output. An action is performed responsive to the output.

### RELATED APPLICATION INFORMATION

This application claims priority to U.S. Patent Application No. 63/468,053, filed on May 22, 2023, and to U.S. Patent Application No. 63/525,757, filed on Jul. 10, 2023, incorporated herein by reference in their entirety.

### BACKGROUND

### Technical Field

The present invention relates to machine learning models and, more particularly, to large language models.

### Description of the Related Art

Large language models, trained on a large amount of text data, can perform well on a variety of downstream tasks. However, training an LLM is expensive, using many processors working in parallel, with large amounts of memory, of a long span of time. This puts the training of an LLM out of reach for all but the largest corporations.

### SUMMARY

A method for configuring a machine learning model includes selecting a head from a set of stored heads, responsive to an input, to implement a layer in a transformer machine learning model. The selected head is copied from persistent storage to active memory. The layer in the transformer machine learning model is executed on the input using the selected head to generate an output. An action is performed responsive to the output.

A system for configuring a machine learning model includes a hardware processor and a memory that stores a computer program. When executed by the hardware processor, the computer program causes the hardware processor to select a head from a set of stored heads, responsive to an input, to implement a layer in a transformer machine learning model. The selected head is copied from persistent storage to active memory. The layer in the transformer machine learning model is executed using the selected head to generate an output. An action is performed responsive to the output.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating a transformer layer with dynamic head selection, in accordance with an embodiment of the present invention;

FIG. 2 is a block/flow diagram of a method for selecting heads for use in a transformer layer, in accordance with an embodiment of the present invention;

FIG. 3 is a block/flow diagram of a method for selecting heads for use in a transformer layer, in accordance with an embodiment of the present invention;

FIG. 4 is a block/flow diagram of a method for training and using a transformer model with head selection, in accordance with an embodiment of the present invention;

FIG. 5 is a block diagram of a healthcare facility that uses a machine learning model with head selection to perform medical record analysis and treatment recommendation, in accordance with an embodiment of the present invention;

FIG. 6 is a block diagram of a computing device that can train a model with head selection for diagnosis and treatment, in accordance with an embodiment of the present invention;

FIG. 7 is a diagram of an exemplary neural network architecture that can be used in a model with head selection, in accordance with an embodiment of the present invention; and

FIG. 8 is a diagram of an exemplary deep neural network architecture that can be used in a model with head selection, in accordance with an embodiment of the present invention.

### DETAILED DESCRIPTION OF PREFERRED EMBODIMENTS

Large language models (LLMs) may be based on a variety of architectures and many combine different types of machine learning model. In particular, some LLMs are based on transformer architectures. Instead of using a large model that executes all of its parameters for every input, weight attention may be used to limit the number of parameters that are executed. Such a model has a lower execution cost per input. Additionally, system memory may be used to hold the bulk of the model, while only the relevant parts may be transferred to processor memory, which reduces the hardware cost associated with training and execution.

A transformer model may include multiple layers, with each layer including a number of heads. The heads perform linear transformations of input values. A model may include a number of heads in every layer of the transformer. During execution, fewer than all of the heads may be executed.

Given the current input to the layer, a prediction may be formed of which heads will be used. This prediction may be made by pooling over all the features of the input vectors for a layer and using a linear layer to project the result to a score that predicts the final performance of the network as a whole for the given input, given that the head corresponding to the score is selected. During training, an epsilon greedy approach may be used to pick the predicted best heads, but some heads may be selected at random a predetermined portion of the time (e.g., 25%). The selected heads may be concatenated and shaped into a matrix that has the same dimensions as the original transformer model.

There may also be a projection back to an embedding dimension toward the end of the layer that is treated the same way. A projection linear layer for the selected heads may be constructed from a store of weights, with the same indices used for the heads.

The values predicted from all the layers of the transformer may be compared to an objective function, with the mean square error of the difference being added to the loss. The LLM and the head utility prediction layers may thereby be trained together using stochastic gradient descent (SGD) and automatic differentiation.

The resulting LLM is a dynamic model, which adapts the parameters that it uses to each input. Attention is performed over the weights of the LLM itself. Some stored weights may thereby become specialized during training, providing specific information or relations from the training corpus that are useful for particular kinds of inputs. A scoring function predicts the utility of including heads. Reinforcement learning is used to determine actions (e.g., the heads to employ). The scoring function can furthermore be extended to include other metrics, such as the time taken to retrieve the weights for a particular head from system memory. The model can thereby be constructed with weights in different locations, such as in processor memory, system memory, and remote storage.

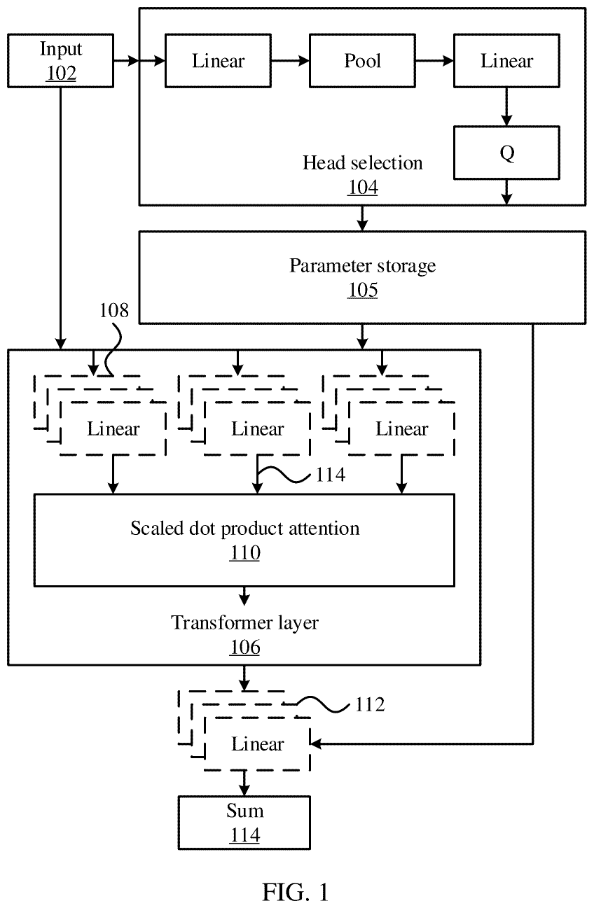

Referring now to FIG. 1, a layer of a transformer-based LLM is shown. An input 102 is provided to the transformer layer 106. This shows just one transformer layer 106 out of a larger LLM, and it should be understood that one or more similar transformer layers 106 may precede or follow the illustrated transformer layer 106, with the output generated by sum 114 being used as the input 102 for the next layer. The input 102 also goes to head selection 104, which uses a policy Q to determine linear layers 108 that the transformer layer 106 will use to process the input.

The transformer layer 106 includes a number of linear heads 108, which include respective sets of weights that are learned during training of the LLM. During use, these heads 108 may be activated by a set of parameters stored in parameter storage 105. The parameters may be transferred from the parameter storage 105 to an active memory, such as the memory of a graphics processing unit (GPU), for use by the transformer layer 106. The transformer layer uses a scaled dot product attention 110 to process the input 102 based on the loaded heads 108, with attention weights 114 being determined as described below.

The output of the transformer layer 106 is similarly processed by a linear unit 112 that takes its parameters from the parameter storage. The output linear unit 112 applies independent linear transformations corresponding to the heads 108 that are selected for the transformer layer 106. Their outputs may then be combined with a sum operation 114.

Head selection 104 may be implemented by a multi-layer neural network that takes the input 102 and transforms it into a score that may be used to rank the stored weights in parameter storage 105. The parameter storage 105 includes weights that may be used in the linear heads 108 and the output layer 112, and may store multiple versions of the weights that can be selected from based on the scores output by head selection 104. These versions may be specialized for different types of input during training and may be selected during inference time.

Head selection 104 may be implemented with a linear layer to project a sequence of input vectors and to perform embedding. A max pooling layer operates in the feature dimension to create a single vector representing the content of the input sequence. A policy function Q takes the vector representation of the sequence and generates a score for every set of parameters in parameter storage 105. During training, these prediction scores may be collected for later use in calculation of a loss function.

Head selection 104 selects the maximum predicted Q value for each group of weights that correspond to each active head position of the transformer layer 106. For example, if there are three active head positions, and twenty-four total stored weight sets, then the choice for each active head will select the highest score out of eight. During training, the model tries to use heads that aren't the current maximum predicted score. This introduces noise into the selection process and forces the model to select some proportion of heads at random. This is an epsilon greedy exploration approach in reinforcement learning, which encourages exploration of the possible actions during training, as opposed to a strict exploitation that would always select the current best score.

The input 102 to the first transformer layer 106 may be a sequence of vectors representing tokens. These tokens may be words or parts of words taken from a source text, such as a sentence or a longer block of text. A token dictionary may be predetermined, so that the input text may be quickly rendered as vectors by looking tokens up in the token dictionary. The first transformer layer 106 transforms the token input into another sequence of vectors, with a one-to-one correspondence between the input vectors and the output vectors. The output of the first transformer layer 106 is used as input 102 to the next transformer layer 106 and this process repeats until the last transformer layer 106 generates its output.

The scores described above may be generated by the policy function Q for every head in storage 105. The policy function may be implemented as a linear layer, where the input to the policy function may be a vector and the output may be a vector of scores, one for each head. The policy network may be trained to predict the expected performance of the model if a particular head is selected. Some heads may be selected at random during training so that the model can see the expected performance of lower scoring heads. The loss for training the policy network may be the difference between the predicted loss, in terms of reconstruction accuracy, and the actual reconstruction loss found by forwarding the model.

The selected parameters are copied from parameter storage 105 into the heads 108 of the transformer layer 106. This may include copying the respective weights from system memory to GPU memory. Parameters for the linear projection layers 112 are further copied. The transformer layer 106 is then fully executed on the input 102 and processing repeats for a next input 102.

During training, the predicted scores and head selections from all the layers are saved. This is compared to a loss function calculated for the model given the training examples and the current training objective. The distance between the loss and the predicted scores may be calculated, with the sum being added to the training loss for a gradient calculation to determine a total loss for a current training example. This learns the original training objective, predicts the performance of using specific heads for specific inputs, and creates specialized heads that are activated for specific inputs.

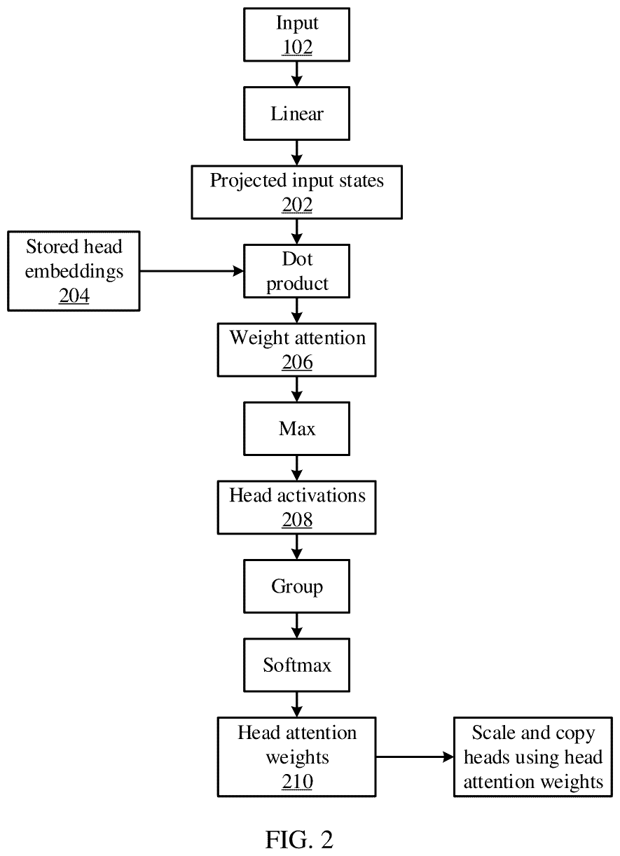

Referring now to FIG. 2, the generation of attention weights 114 is shown. A linear layer projects input 102 to projected input states 202 in an appropriate latent space. A dot product is performed between the projected input states 202 and the stored head embeddings 204, which are the same as the learned weight attention embeddings, with a respective vector for each head. This provides a weight attention matrix 206 as S×WA, where S is the number of input tokens and WA is a matrix, with values resulting from the dot product of each pair of the projected input states and the stored head embeddings. The matrix may have dimensions dictated by the number of input states and the number of head embeddings. The maximum of each column is taken to create a single vector of stored head activations, with a value for each stored head embedding.

The sequence dimension is pooled, for example using a max or mean function, to produce a head activation vector 208 that has a length of WA, representing the strength of activation for each stored head. The vector is reshaped into a matrix, where the rows correspond to groups for each head to provide a M×N matrix, where N is a number of heads in the active path of the model, corresponding to a number of groups, and where M is the number of embeddings per group. A softmax operation is performed over the M dimensions, which has the effect of normalizing the weights for each group corresponding to each head. Other normalization methods may be used instead of softmax. This produces attention weights 210 for each stored head. Weights for the transformer model, which are used to process inputs, may be generated for each head by multiplying each of the stored weights in a corresponding group by the attention weights 210 and summing the result. The stored weights are parameters that are learned during training, with a set of stored weights for each head in the active path of the model. The resulting matrix for each head includes Q, K, and V matrices for the head. The matrices for all heads are interleaved into one matrix. The head attention weights are used to scale and copy stored heads from parameter storage 105 for use in the transformer layer 106.

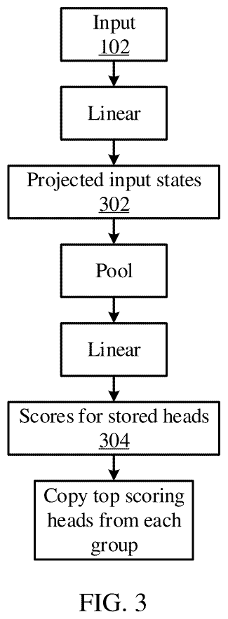

Referring now to FIG. 3, the use of a policy function to select heads is shown. As above, a linear layer projects input 102 to projected input states 302 in an appropriate latent space. The projected states are max pooled over a feature dimension to create a single vector that represents the content of the input sequence 102. A linear layer is used to implement the policy network Q, which generates scores for the stored heads 304 based on the projected input states 302.

The maximum predicted Q value for each group of weights may be selected to correspond to the active head positions for the transformer layer 106. For example, if there are three active head positions, and twenty-four total stored sets of weights, then the choice for each active head is one out of eight. As noted above, the Q network may be trained using an epsilon greedy reinforcement learning approach, where exploration and exploration policies are managed to control the use of unexplored states.

The selected heads are copied into the positions for the heads for the forward pass of the transformer layer 106. This operation may move heads from CPU memory or other storage into the video memory of a graphics card. This process may be performed for each transformer layer 106 in a transformer model.

During training, the predicted scores and head selections are collected from all of the transformer layers 106. These may be compared to the error calculated for the model given training examples and the training objective. The distance between the predicted scores and this training loss may be determined, and the sum may be added to the training loss for gradient computation to generate a total loss for a given training example. Through automatic differentiation and stochastic gradient descent, the original training objective, performance predictions for the specific heads on specific inputs, and specialized heads for specific inputs may be learned.

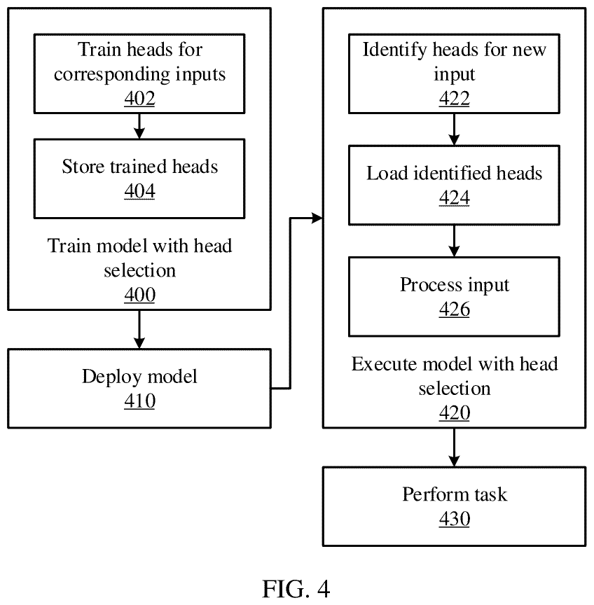

Referring now to FIG. 4, a method for training and using a transformer model is shown. Block 410 begins by training the model with head selection. As described above, each transformer layer 106 in the model has a set of heads, implemented by the linear layers 108, which may be altered in response to training data. In addition to training using backpropagation, the training furthermore creates specialized heads for corresponding inputs in block 402, which are stored in persistent storage 105 by block 404. This training makes it possible to select particular heads responsive to the input.

Block 410 then deploys the model to a target environment. For example, block 410 may transfer the model and the stored heads to a healthcare facility, where it may be used to aid in medical decision making. Block 420 executes the model with head selection responsive to inputs. Block 422 identifies the stored heads that are appropriate for a given input and block 424 loads the identified heads into memory for use in a transformer layer 106. Block 426 then process the input, for example applying the input to the transformer model in a feed-forward operation to generate an output.

Based on the output of the transformer model, block 430 performs a task. Such a model may be used to assist with explaining medical records and filling in forms. Inputs may include patient history and image data and may be used to diagnose illnesses, for example using the patient's health information and an image of a tissue sample to diagnose whether a given tissue sample shows evidence of a disease.

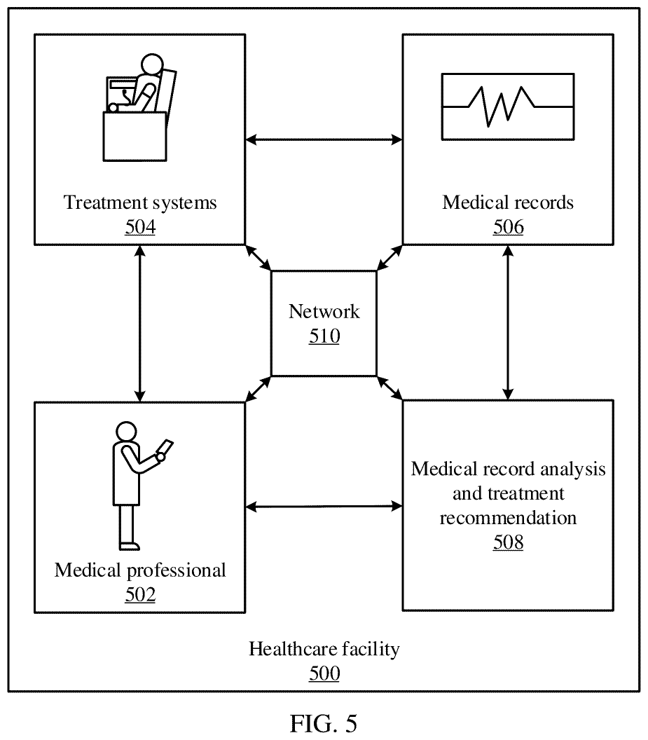

Referring now to FIG. 5, a diagram of information extraction is shown in the context of a healthcare facility 500. Medical record analysis and treatment recommendation 508 may be used to process information relating to genes taken from a patient's tissue sample. The medical record analysis and treatment recommendation 508 may review stored health information about a user, for example including their medical history, test results, and tissue sample images, to produce a diagnosis and recommended treatment. This diagnosis informs patient treatment and medical decision-making.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Based on information drawn from the medical record analysis and treatment recommendation 508, the medical professionals 502 may then make medical decisions about patient healthcare suited to the patient's needs. For example, the medical professionals 502 may make a diagnosis of the patient's health condition and may prescribe particular medications, surgeries, and/or therapies.

The different elements of the healthcare facility 500 may communicate with one another via a network 510, for example using any appropriate wired or wireless communications protocol and medium. Thus medical record analysis and treatment recommendation 508 receives information about a tissue sample from medical professionals 502, from treatment systems 504, from medical records 506, and updates the medical records 506 with the output of the GNN model. The medical record analysis and treatment recommendation 508 may coordinate with treatment systems 504 in some cases to automatically administer or alter a treatment. For example, if the medical record analysis and treatment recommendation 508 indicates a particular disease or condition, then the treatment systems 504 may automatically halt the administration of the treatment.

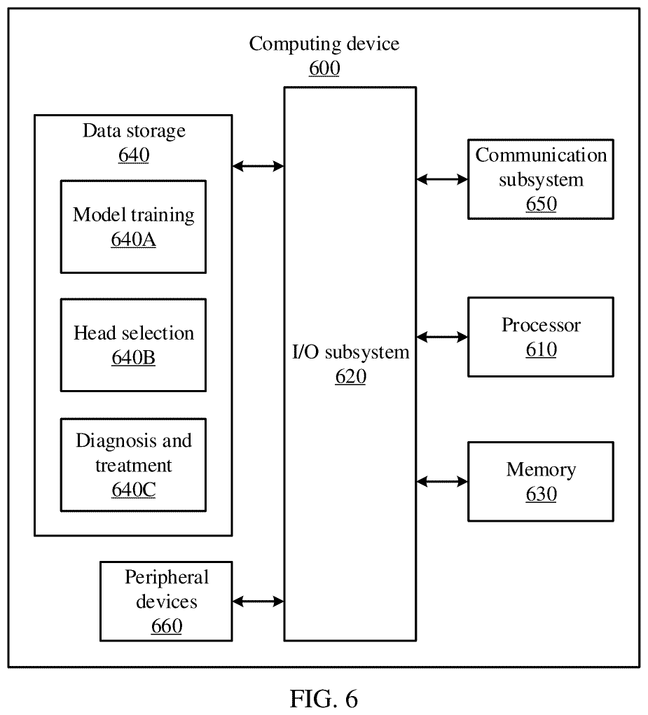

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for training a model, 640B for selecting and storing model heads, and/or 640C for performing diagnosis and treatment. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the computing device 600 are readily contemplated by one of ordinary skill in the art given the teachings of the present invention provided herein.

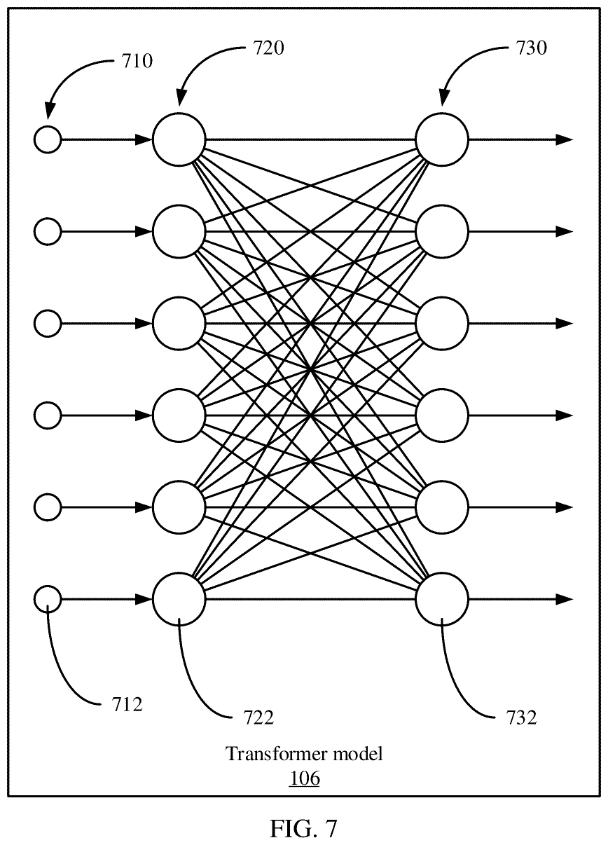

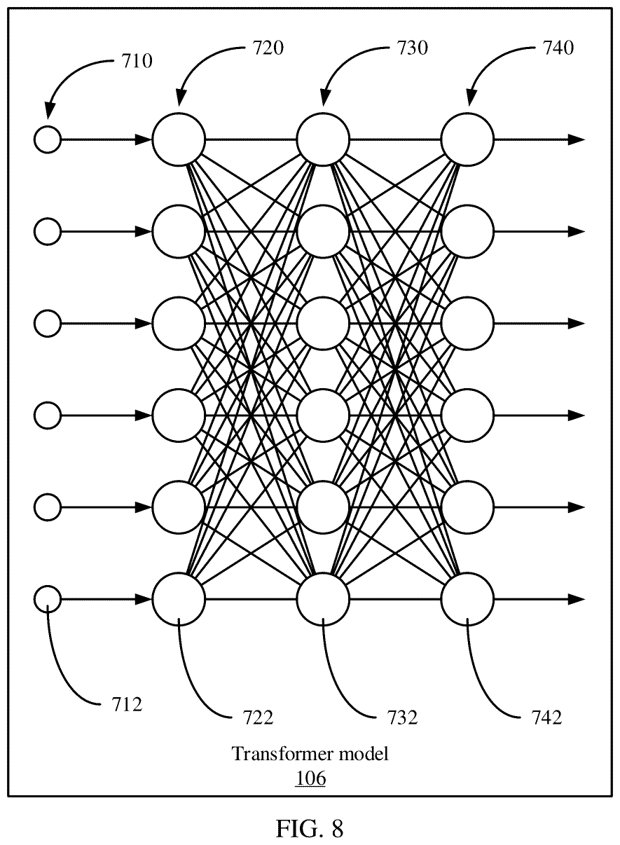

Referring now to FIGS. 7 and 8, exemplary neural network architectures are shown, which may be used to implement parts of the present models, such as the transformer layer 106. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, W₂, . . . Wₙ₋₁, Wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor—or computing element—based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for configuring a machine learning model, comprising:
selecting a head from a plurality of stored heads, responsive to an input, to implement a layer in a transformer machine learning model;
copying the selected head from persistent storage to active memory;
executing the layer in the transformer machine learning model on the input using the selected head to generate an output; and
performing an action responsive to the output.

2. The method of claim 1, wherein selecting the head includes processing the input with a policy network that generates a score for each of the plurality of stored heads and selecting a head from the plurality of stored heads having a highest score.

3. The method of claim 2, wherein the generated scores indicate expected performance for the respective plurality of stored heads.

4. The method of claim 2, wherein the policy network is implemented as a linear neural network layer.

5. The method of claim 1, wherein selecting the head includes determining a weight attention matrix based on a dot product between embeddings of the plurality of stored heads and an embedding of the input.

6. The method of claim 5, wherein selecting the head further includes pooling the weight attention matrix to generate a strength of activation for each of the plurality of stored heads.

7. The method of claim 5, wherein selecting the head includes generating head weights for each of the plurality of stored heads by multiplying stored weights in a corresponding head group by attention weights and summing a result.

8. The method of claim 1, wherein the input includes patient medical information and wherein the output includes a prediction of disease to aid in medical decision making.

9. The method of claim 8, wherein the patient medical information includes the patient's medical history and an image of a tissue sample.

10. The method of claim 1, wherein the action includes automatically altering a patient's treatment.

11. A system for configuring a machine learning model, comprising:
a hardware processor; and
a memory that stores a computer program which, when executed by the hardware processor, causes the hardware processor to:
select a head from a plurality of stored heads, responsive to an input, to implement a layer in a transformer machine learning model;
copy the selected head from persistent storage to active memory;
execute the layer in the transformer machine learning model on the input using the selected head to generate an output; and
perform an action responsive to the output.

12. The system of claim 11, wherein the computer program further causes the hardware processor to process the input with a policy network that generates a score for each of the plurality of stored heads and selecting a head from the plurality of stored heads having a highest score.

13. The system of claim 12, wherein the generated scores indicate expected performance for the respective plurality of stored heads.

14. The system of claim 12, wherein the policy network is implemented as a linear neural network layer.

15. The system of claim 11, wherein the computer program further causes the hardware processor to determine a weight attention matrix based on a dot product between embeddings of the plurality of stored heads and an embedding of the input.

16. The system of claim 15, wherein the computer program further causes the hardware processor to pool the weight attention matrix to generate a strength of activation for each of the plurality of stored heads.

17. The system of claim 15, wherein the computer program further causes the hardware processor to generate head weights for each of the plurality of stored heads by multiplying stored weights in a corresponding head group by attention weights and summing a result.

18. The system of claim 11, wherein the input includes patient medical information and wherein the output includes a prediction of disease to aid in medical decision making.

19. The system of claim 18, wherein the patient medical information includes the patient's medical history and an image of a tissue sample.

20. The system of claim 11, wherein the computer program further causes the hardware processor to automatically alter a patient's treatment.
